(19) United States
(12) Patent Application Publication
(10) Pub. No.: US 2022/0163520 A1
(43) Pub. Date: May 26, 2022

(54) APPARATUS, METHOD AND KIT FOR DETECTION OF VON WILLEBRAND FACTOR AND FACTOR VIII

(71) Applicant: INDIAN COUNCIL OF MEDICAL RESEARCH, New Delhi (IN)

(72) Inventors: Priyanka Arun KASATKAR, Parel Mumbai (IN); Shrimati Dharmapal SHETTY, Parel Mumbai (IN)

(73) Assignee: INDIAN COUNCIL OF MEDICAL RESEARCH, New Delhi (IN)

(21) Appl. No.: 17/440,677

(22) PCT Filed: Mar. 19, 2020
(86) PCT No.: PCT/IN2020/050260

§ 371 Date: Sep. 17, 2021

(30) Foreign Application Priority Data
Mar. 19, 2019 (IN) 201911010626

(51) Int. Cl. G01N 33/543; G01N 33/553; G01N 33/558

(52) U.S. Cl. CPC G01N 33/54388; G01N 33/54346; B82Y 5/00; G01N 33/558; G01N 2333/755; G01N 33/553

(57) ABSTRACT

The present invention provides a rapid, specific, user friendly and cost effective lateral flow immunoassay based apparatus, method and kit for the detection of FVIII:Ag and VWF:Ag from human plasma samples. The LFIA based method and kit of the present invention can be used for the diagnosis of newly undiagnosed patients with bleeding history, menorrhagia cases, gynecological complications, differential diagnosis of Hemophilia A and VWD, recovery of factors in the transfused patient etc.

### FIELD OF THE INVENTION

The present invention provides a rapid, user friendly and cost-effective lateral flow immunoassay (LFIA) based apparatus, method and kit for the detection of Von Willebrand factor and Factor VIII.

### BACKGROUND OF THE INVENTION

There are many different types of bleeding disorders (inherited as well as acquired), of which Von Willebrand disease (VWD) and Hemophilia are most common. Hemophilia A, an inherited single gene disorder is a genetic disorder caused by defective clotting protein-factor VIII (inherited in an X-linked recessive pattern). Hemophilia is reported with an incidence of 1 per 10000 births and according to Hemophilia Federation of India data, the reported number of patients with Hemophilia A is 20000 while the estimated prevalence could be around 100000 patients. People with severe Hemophilia A often present with prolonged bleeding (bleeds can occur internally into joints and muscles, or externally from minor cuts, dental procedures or trauma). Patients with mild/moderate Hemophilia A generally experience bleeding only after serious injury, trauma or surgery. In many cases, mild Hemophilia is not diagnosed until an injury, surgery or tooth extraction.

Von Willebrand disease (VWD) is the commonest autosomal bleeding disorder affecting both the sexes. Patients with VWD often present with mucosal bleeds like ecchymosis, nose bleeds, prolonged bleed after trivial trauma etc. Women with VWD often experience menorrhagia, heavy menstrual periods, and hemorrhage after childbirth. As reported by Kasatkar P et al., 2014, the estimated prevalence of VWD reported in western countries is 1% of population i.e. expected VWD should be approx. 1 crore (135 crores—India's population) due to consanguinous marriages and common mutations seen in few communities. Patients manifest as spontaneous or trauma-induced haemorrhagic episodes leading to premature mortality in untreated patients or patients with sub-optimal treatment.

In India, inherited bleeding disorders (VWD/Hemophilia A) largely remain undiagnosed for varied reasons i.e. mild bleeding symptoms, inadequate awareness of the disease in society as well as treating physicians and paucity of diagnostic facilities. The main medication to treat Hemophilia A is concentrated FVIII product or recombinant factor products, the latter developed in a laboratory through the use of DNA technology. While plasma derived FVIII products are still available, approximately 75% of the Hemophilia community takes the recombinant FVIII product. These factor therapies are infused intravenously through a vein in the arm or port in the chest. There is a high risk of alloantibody development in severe Hemophilia A patients after repeated transfusion with factor concentrates. The VWF rich factors are scarcely available and are very expensive. So, VWD patients are mostly transfused frozen plasma or cryoprecipitate which poses the risk of transfusion transmitted diseases.

A rapid and accurate diagnosis is critical in these patients, as early therapy can be life-saving. There are few comprehensive centers in India which have the required laboratory facilities for the diagnosis of Hemophilia, von Willebrand disease (VWD) and other coagulation factor deficiency disorders. The existing methods (coagulation based tests that are standard tests) for the detection of FVIII:Ag/VWF:Ag are time consuming, expensive and need fresh blood sample, technical expertise, sophisticated instrument and a panel of tests are required to confirm the diagnosis. In case of bleeding patients, the waiting time is long and can extend up to few days for the specific tests such as ELISA in which samples are processed in batches. A rapid and accurate diagnosis is critical in these patients, as early therapy can be life-saving.

A “point of care” test is an investigation done at the time of consultation with immediate availability of results to make immediate decision about patient care. In recent times, lateral flow immunoassay (LFIAs) based point of care (POC) testing is extensively used in pregnancy testing, detecting the contaminants in water, food and diagnosing the infectious diseases too with accuracy. So far, no commercial rapid test kit is available for specific diagnosis of any of the aforesaid common bleeding disorders.

The present invention thus aims to provide a rapid, specific, user friendly and cost effective lateral flow immunoassay based apparatus, method and kit for the detection of FVIII:Ag and VWF:Ag from human plasma samples. The inventors of the present invention have established a novel multiplex architecture of the LFIA membrane with a common absorbent pad, simple POC technique for the detection of VWF:Ag and FVIII:Ag from the patient's plasma sample. The kit of the present invention is also capable of simultaneously detecting FVIII:Ag and VWF:Ag from human plasma samples in a single step within 30 minutes of sample collection. The VWF LFIA assay of the present invention is 99% specific with 99% accuracy when assessed with VWD patients and other known deficiency patient samples. Whereas, FVIII assay of the present invention is 98% specific with 96% accuracy till date when tested with the available plasma samples of Hemophilia A. The kit of the present invention is economically efficient and requires quite reduced amount of sample (50 μL) compared to known techniques (1-2 mL).

### OBJECTIVES OF THE INVENTION

An important objective of the present invention is to provide an apparatus for the detection of FVIII (Hemophilia A) and VWF (VWD) in human plasma samples.

Another objective of the present invention is to provide a lateral flow immunoassay method for the simultaneous detection of Hemophilia A (FVIII:Ag) and von Willebrand disease (VWF:Ag) in human plasma samples.

Yet another objective of the present invention is to provide a uniplex or multiplex kit for separately or simultaneously detecting FVIII and/or VWF.

### BRIEF DESCRIPTION OF FIGURES

FIG. 1 shows gold nanoparticles (GNPs) synthesized using different protocols i.e. different concentrations of gold chloride (0.5-1 mM), citrate (0.2-2%) and capping agent.

FIG. 2 illustrates size distribution report (small size GNPs synthesized-20 nm) by different protocols and its analysis.

FIG. 3 provides Zeta potential of the GNPs analyzed by MALVERN nano series.

FIG. 4 shows High Resolution Transmission Electron Microscopic (HR-TEM) images of the GNPs synthesized. a) Spherical GNPs of 10-17nm synthesized by Citrate reduction method b) Citrate stabilized TEM bright field images of the gold nanoparticles c) Diffraction of Au-NP observed using TEM d) Lattice fringes of Au nanoparticle by HR-TEM.

FIG. 5 shows the effect of pH on the GNPs synthesized.

FIG. 6 shows the architecture of the multiplex kit for simultaneous detection of both FVIII and VWF.

FIG. 7 shows the interior components of the membranes assembled in the multiplex VWF- FVIII combo kit.

FIG. 8 shows test and control lines for both the proteins FVIII and VWF.

FIG. 9 shows strip with severe Hemophilia sample and absence of test line in FVIII side.

FIG. 10 shows strip with severe VWD sample and absence of bands on test lines in both FVIII and VWF ends.

FIG. 11 provides the flow diagram showing approach for investigation of patients with bleeding disorders.

### SUMMARY

The present invention provides a rapid, specific, user friendly and cost effective lateral flow immunoassay based apparatus, method and kit for the detection of FVIII:Ag and VWF:Ag from human plasma samples. The LFIA based method and kit of the present invention can be used for the diagnosis of newly undiagnosed patients with bleeding history, menorrhagia cases, gynecological complications, simultaneous detection of Hemophilia A and VWD, recovery of factors in the transfused patient etc.

The kit of the present invention is also capable of simultaneously detecting FVIII:Ag and VWF:Ag from human plasma samples in a single step within 30 minutes of sample collection. The VWF LFIA assay of the present invention is 99% specific with 99% accuracy when assessed with VWD patients and other known deficiency patient samples. FVIII assay of the present invention is 98% specific with 96% accuracy till date when tested with the available plasma samples of Hemophilia A. The kit of the present invention is economically efficient and requires quite reduced amount of sample (50 μL) compared to known techniques (1-2 mL).

Detection of VWF: Ag is essential for the differential diagnosis in Hemophilia A and VWD. The presence or the absence of the band on the test zone can help in a quick diagnosis just by visual observation and aid in the treatment of bleeding patients with specific products for each of these two disorders. Also, this immediate testing reduces both perioperative blood loss and the rate of transfusion of allogeneic blood products in the patients. Thus, the kit and method of the present invention would help in immediate diagnosis of VWD and Hemophilia A and can be made available easily even in remotest of areas in the laboratories with the basic facilities i.e., centrifuge and pipette only. In this way, the present invention would help reduce the mortality rate in bleeding patients.

### DETAILED DESCRIPTION

The details of one or more embodiments of the invention are set forth in the accompanying description below including specific details of the best mode contemplated by the inventors for carrying out the invention, by way of examples. It will be apparent to one skilled in the art that the present invention may be practiced without limitation to these specific details.

The present invention provides rapid, user friendly and cost-effective lateral flow immunoassay-based apparatus, method and kit for the detection of FVIII:Ag and VWF:Ag from human plasma samples. More specifically, a novel architecture of the LFIA membrane with a common absorbent pad for the simultaneous detection of coagulation proteins VWF and FVIII is provided.

Material and Methods

A. Sample Collection and Processing

Blood samples were collected in tubes containing tri sodium citrate (3.2%) (9cc blood: lcc anticoagulant). The citrated samples were centrifuged at 4000 rpm for 15 minutes at 4° C. to obtain platelet poor plasma (PPP) for analysis. The labeled aliquots of PPP were stored at −70° C. for further analysis.

B. Patient and Controls

Blood samples were collected from 100 VWD and 168 Hemophilia A patients referred from various Municipal and Private Hospitals in India. Though majority of the cases i.e., 70% patients come from western India, others are from north, south and eastern parts of India. Samples from 20 healthy controls who gave no history of bleeding or family history of bleeding were collected and stored in aliquots at −70° C. Informed consent was obtained from the patients or their relatives prior to blood collection.

Laboratory Evaluation and Diagnosis

A. Sample Collection in Citrate Vacutainer

The blood samples were kept at room temperature for 15 minutes prior to centrifugation for the coagulation studies. The blood samples were centrifuged at 500-600 rpm (depending on the platelet count) at 25° C. to obtain Platelet Rich Plasma (PRP). PRP was separated after centrifugation and the samples were spun again at 4000 rpm for 15 minutes at 4° C. Platelet function assay was performed with PRP to rule out platelet disorder or VWD by platelet induced ristocetin aggregation assay.

After second centrifugation, the Platelet poor plasma (PPP) was separated and used for screening and coagulation analysis that involves the specific factor assays (depending on intrinsic or extrinsic pathway defects) to arrive at the final diagnosis.

B. Screening Tests

The most common tests such as activated partial thromboplastin time (APTT) prothrombin time (PT), and optionally either a fibrinogen level or thrombin time (TT) were performed. An initial hemostasis laboratory evaluation usually includes a platelet count and complete blood count (CBC).

The existing standard coagulation-based protocols/techniques for the diagnosis of Hemophilia A or VWD need specific tests using the specific deficient plasma (very expensive) by APTT based assay. The detection of FVIII:Ag (Commercial kits- ELISA method), FVIII:C (by screening coagulation method using factor VIII deficient plasma), VWF:Ag (Commercial kits—ELISA/Automated instrument) and the alloantibodies to FVIII (Screening coagulation by 2 hour incubation mixing studies and quantitation by Nijmegen-Bethesda assay) and alloantibodies to VWF (Mixing studies by platelet aggregometry/Immunoelectrophoresis, automated machine assay) as standardized in the laboratory were assayed accordingly for all the samples to be used for LFIA.

C. Major Steps in LFIA
Antibodies against FVIII and VWF were procured from different sources (Sigma Aldrich, USA; Dako, Denmark; Novus Biologicals, USA; Abcam, USA; Raybiotech Life, USA; cloud clone corp, USA; Merck, USA and Thermo fisher Scientific, USA). Different monoclonal as well as polyclonal antibodies with different epitope specificities of both FVIII and VWF were selected for the detection of FVIII:Ag and VWF:Ag from the patient's plasma. The secondary antibodies were selected accordingly for each assay. For FVIII strips, several combinations of monoclonal and polyclonal antibodies were tried from different sources which had different affinity towards different domains of factor VIII protein molecule. Combination of two different monoclonal antibodies showed better results when tagged for conjugation with gold nanoparticles and other coated on reaction membrane. Though VWF is a complex multimeric glycoprotein, polyclonal antibody raised towards different epitopes of VWF molecules (Dako, Denmark) was successfully used for the LFIA developed.
b. LFIA membranes: Different lateral flow grade membranes like sample pad, conjugate release matrix, reaction membrane and absorbant pad were selected for the study. These LFIA grade membranes were purchased from Advanced Microdevices, Ambala, India. Selection of the membranes depends on the assay with various parameters like pore size/wicking rate, affinity for antigen-antibody reaction and migration speed on membranes provided with or without buffers.
c. The most reliable, widely used gold nanoparticles were synthesized in the laboratory using different citrate reduction methods as mentioned below. During the synthesis process, all the glasswares used in the experiments were deep cleaned by aqua regia (conc. HNO₃ and conc. HCl), dried and then rinsed with double distilled water before using for nanoparticle synthesis.

D. Gold nanoparticle (GNP) synthesis

The citrate reduction process was used for synthesis of the gold nanoparticles. The reduction of a tetrachloroauric acid (HAuCl₄) was initiated by trisodium citrate (Na₃C₆H₅O₇) by injecting specified amount of preheated trisodium citrate solution to a boiled gold solution in a beaker. The mixture liquid was vigorously stirred by Teflon coated magnetic bars. The color of the solution changed gradually from transparent light yellow, dark black, and finally to the characteristic wine red, which indicated the formation of gold nanoparticles. Different concentrations of trisodium citrate were used during the standardization to reduce 1 mM HAuCl₄ and 0.5 mM HAuCl₄. Amounts of tetrachloroauric acid and trisodium citrate were varied to achieve different particle size distributions. Different agents like tannic acid and formalin were also used during the synthesis to obtain smaller size and uniform gold nanoparticles.

The 20 nm sized gold nanoparticles were synthesized by injecting 38.8 mM Trisodium citrate in boiling 0.5 mM HAuCl4 in 100 ml of MilliQ water on the magnetic stirrer. The color of the solution changes from transparent to slight yellow to grey to purple and then finally red characterized by visual wine red colour indicating formation of gold nanoparticles. After a defined time, the liquid was cooled to room temperature and stored in dark for further analysis.

After synthesizing the gold nanoparticles, the spectral analysis was done using spectrophotometer. Characterization of gold nanoparticles was done at the Institute of Chemical Technology, Matunga for Dynamic Light Scattering Analysis and at Sophisticated Analytical Instrument Facility (SAIF) IIT Bombay, Powai for TEM imaging.

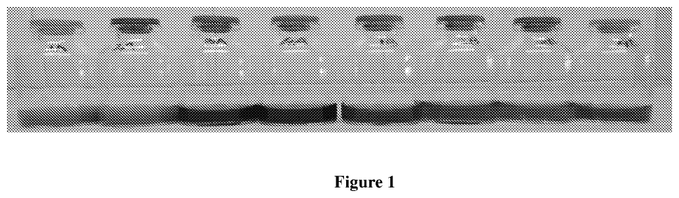

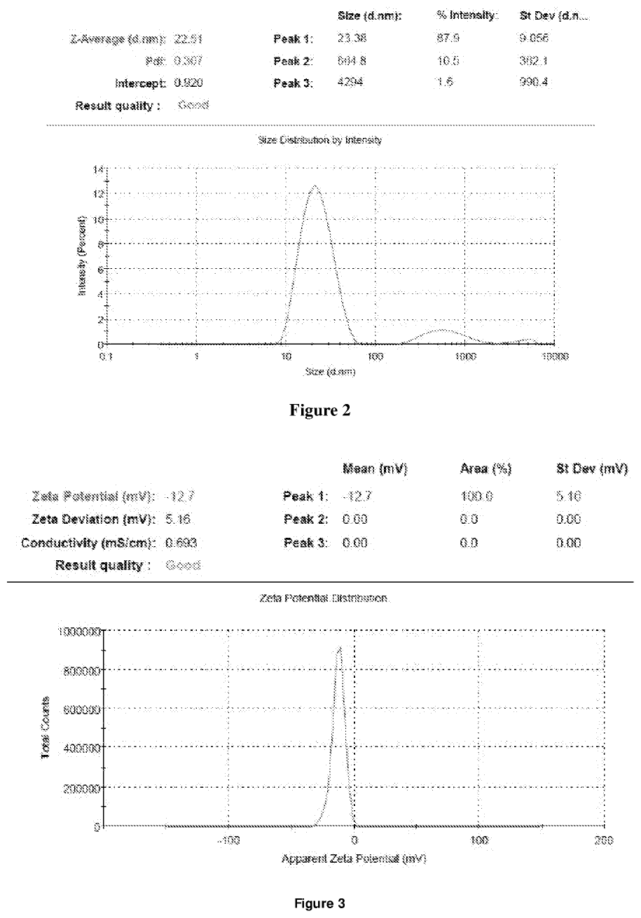

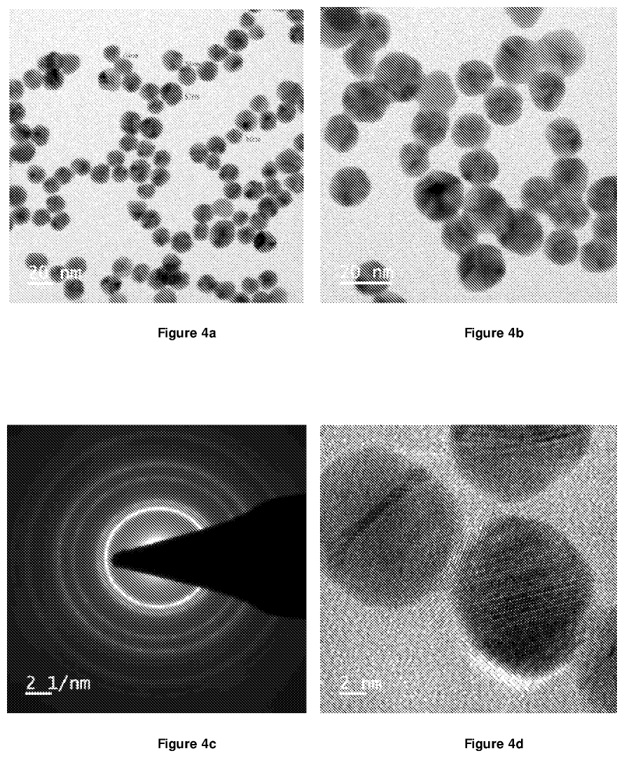

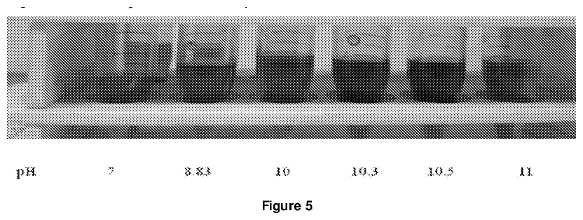

E. Characterization of gold particles
a. Visual Inspection: Formation of GNPs resulted in a sharp color change from yellow to wine red. Thus, a visual inspection was routinely performed (FIG. 1).
b. UV-Vis Spectra Analysis: UV-Visible spectra of GNPs by reduction of Chloroauric acid in aqueous solution was recorded in Tecan- Infinite M200PRO.
c. Dynamic Light Scattering (DLS) Analysis: DLS was performed in MALVERN Nano Series in Institute of Chemical Technology (ICT, Matunga) to measure the hydrodynamic diameter and zeta potential of the GNPs synthesized. Several parameters of the synthesized GNPs with different size, dispersity and concentration were studied for each sample from all the above protocols using Zeta sizer (FIG. 2-3). The GNPs synthesized by citrate reduction exhibit a zeta potential that is negative (−15 mV±3 mV).
d. Transmission Electron Microscopic (TEM) Measurement: The samples for TEM analysis were prepared by drop-casting the GNPs solution on a carbon-coated copper TEM grid. Before casting to the grid, the GNPs solution was sonicated in a sonicator for 15 minutes. As the GNPs were very small in size (<20 nm) high resolution TEM for better resolution was done on a high-resolution electron microscope (HRTEM: PHILIPSCM 200) operating at an accelerating voltage of 200 kV with resolution: 2.4A° in Sophisticated Analytical Instrument Facility (SAIF) HT Bombay, Powai (FIG. 4).

F. Testing the Stability of Gold Nanoparticles at Different pH

GNPs synthesized in the laboratory were then estimated for their stability at different pH. The pH was adjusted with 2% potassium carbonate (K₂CO₃) with varying pH values from 5.5-12.

The GNPs were observed to be stable at a pH between 6.5 to 10.3 while reduced with further increase in the pH (10 onwards) value. UV-Vis spectral analysis for the determination of both size and concentration of gold nanoparticles was done. The analysis was done in Tecan-Infinite M200PRO at different wavelength and the samples are scanned for γmax. The surface plasmon resonance (spr) was clearly visible as a peak in the range between 520 nm and 580 nm

Standardisation: Several parameters like selection of primary and secondary antibodies (monoclonal as well as polyclonal) on the test and control zone, different methods of conjugating antibodies on gold nanoparticles, storage conditions for incubation and drying were studied and optimized.
a. Conjugation—Different strategies of conjugating antibodies with gold nanoparticles such as passive adsorption, covalent bonding were used. Protocols were established using several commercial polyclonal and monoclonal antibodies at the concentration of 0.5-2 mg/ml from different sources. Different concentrations of antibodies for conjugation with GNPs were tried. The samples were incubated at different conditions like room temperature and 4 degrees. Primary as well as secondary antibodies from several sources like Sigma Aldrich, USA; Dako, Denmark; Novus Biologicals, USA; Abcam, USA; Raybiotech Life, USA; Cloud Clone Corp, USA; Merck, USA and Thermo fisher Scientific, USA etc. were used. Different combination of monoclonal as well as polyclonal antibodies with different epitope specificities for both FVIII and VWF were used.
b. Different buffers, concentration of blocking agents and detergents are optimized in the laboratory. Tris, Borate, PBS buffer were tried for standardization with varying concentration of blocking agents selected from bovine serum albumin (BSA), Polyethylene glycol (PEG) and gelatin.
c. LFIA Membranes:
   Sample pad—Glass fiber membrane with varying thickness of 0.35 mm, 0 6 mm, pretreated with and without buffer and detergent were used.
   Conjugate release matrix—Special matrix having capability of releasing the conjugate after drying on membrane with different blocking agents, buffers and detergent to maintain pH, wicking time 18-48 seconds were used.
   Reaction matrix—The actual reaction nitrocellulose (NC) membranes available in different pore size membranes −5 μ, 8 μ, 10 μ, and 15 μ were used. Different combinations and concentration of primary as well as secondary antibodies were tried on the test zone and control zone on the nitrocellulose membrane.
   A semi-automated cutter is used to cut the strips to the required size (3-4mm) The different parts of the membranes (Sample pad, Conjugate release matrix, Reaction matrix and Absorbant pad) were assembled manually and then fixed in a plastic cassette in an aluminium pouch with a dessicant in it.

In an embodiment, the present invention provides a uniplex kit for detection of VWF or FVIII.

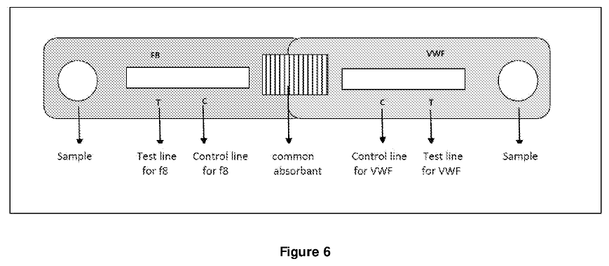

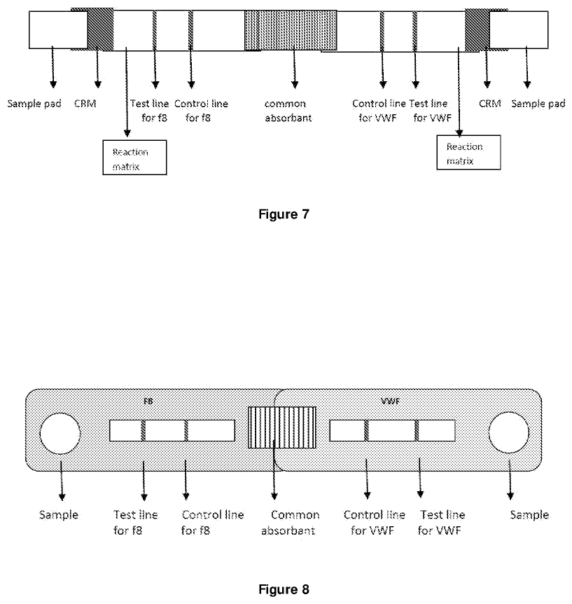

In another embodiment, the present invention provides a 2 in 1 multiplex kit for simultaneous detection of VWF as well as FVIII within 15 minutes wherein the design has a two-way display for the two proteins to be detected. There are two different windows with two different reaction membrane and a common absorbant pad placed vertical to each other illustrated by protein label on it (FIGS. 6 and 7).

In an embodiment, the present invention provides a point-of-care apparatus, wherein the apparatus is in the form of a lateral flow immunoassay-based strip for detection of FVIII and/or Von Willebrand factor (VWF) in a sample comprising:
a) a sample pad;
b) a conjugate pad;
c) a reaction matrix and
d) a common absorbant pad

wherein the strip is fixed in a plastic cassette and comprises gold nanoparticles in the size range of 10-20 nm, primary antibodies and/or secondary antibodies specific to VWF/FVIII and lateral flow grade membranes.

In an embodiment, the apparatus of the present invention is used for separately or simultaneously detecting VWF and/or FVIII.

In another embodiment, the sample pad is made up of glass fiber or polyester membrane with varying thickness of 0.35mm-0 6mm and is optionally pretreated with a buffer and a detergent.

In an embodiment, the buffer is selected from Phosphate buffered saline (PBS), Borate or Tris buffer with varying content of blocking agents for enhancing the stability and releasing the gold conjugate from the conjugate pad, ensuring the uniform movement of gold nanoparticles tagged antibodies and samples on the strip.

In an embodiment, the blocking agent is selected from bovine serum albumin (BSA), Polyethylene glycol (PEG) and gelatin.

In a further embodiment, the apparatus is kept in a sealed aluminium pouch with the desiccant between 4-10° C.

In yet another embodiment, the conjugate pad comprises a membrane made up of a material selected from glass fiber, cellulose filters and surface treated polyester or polypropylene filters capable of releasing the conjugate after drying on said membrane.

In a further embodiment, the membranes are optionally treated with one or more blocking agents, buffers and detergents to maintain a pH between 7-9 with a wicking time between 18-48 seconds.

In still another embodiment, the conjugate comprises antibodies conjugated with gold nanoparticles.

In another embodiment, the reaction matrix comprises nitrocellulose (NC), cellulose or nitrocellulose acetate membranes of pore sizes between 5 μ to 15 μ.

In yet another embodiment, the present invention provides a uniplex or multiplex kit for detection of Von Willebrand factor (VWF) and/or FVIII in a sample comprising:
(i) A strip of the present invention;
(ii) A photo-strip meter or reader that would directly measure the VWF and/or FVIII concentrations from the strip; and
(iii) an instruction/operation manual.

In a further embodiment, the strip reader has dilutions of the known standard in the form of photo print to compare the intensity of the patients strip to the standard provided with the kit.

In another embodiment, the present invention provides a method for preparing the point-of-care apparatus of the present invention, comprising:
a) preparing a sample pad by pretreating a glass fiber or polyester membrane with a buffer and a detergent;
b) conjugating gold nanoparticles (GNPs) with antibodies to make the conjugate pad;
c) selecting a reaction matrix of pore size between 5 μ to 15 μ and an absorbant pad made of high-quality cellulose fiber; and
d) assembling the membranes in the order: Sample pad, Conjugate pad, Reaction matrix and absorbant pad with the overlap so as to obtain a strip and packing the same in a plastic cassette, in an aluminium pouch with a dessicant.

In yet another embodiment, a lateral flow immunoassay based (LFIA) method for detection of Hemophilia and/or Von Willebrand Factor (VWF) in a sample comprising:
a) adding about 50-70 μ of the sample on the sample pad (1) through a cassette window (2) with the help of dropper provided with the kit or pipette;
b) allowing the sample to run by capillary action through the membrane for about 5 to 15 minutes; and
c) visually detecting a red color band.

In another embodiment, the sample is a plasma sample from a subject.

### EXAMPLES

The following examples and advantages are provided for the purpose of illustration and are not intended to limit the scope of the present invention.

### Example 1

### Conjugation of GNPs with Antibodies

The pH of the synthesized ˜20 nm GNPs was adjusted to 10.2 (for Sigma Commercial GNPs the optimum pH was 7.4) using freshly prepared 2% K₂CO₃ solution. To this 10 ml of GNPs 7 μ of polyclonal Rabbit anti-Human VWF antibody (Dako, Denmark) was added and incubated for 1 hour with gentle stiffing at room temperature (25-30° C.).

Freshly prepared blocking BSA buffer (0.1M Tris-HCl with 1% BSA) of pH 8.0 was used. Only fresh MilliQ water to be used in the preparation of reagents/buffers. The pH of the buffer was adjusted with freshly prepared 2% K₂CO₃ solution. To said incubated mix (1 hour incubated with gentle stirring in tube wrapped with aluminium foil, since GNPs are photosensitive), blocking the conjugation reaction with 1-3% BSA at room temperature (25° C.-30° C.) for 30 minutes was done. The conjugated Ab-GNPs solution was then aliquoted in sterile 1 ml tubes and centrifuged at 10000 rpm for 30 minutes at 10° C. Supernatant was discarded, add 1 ml of washing buffer, mix well and centrifuge again. These washing steps were repeated.

The pellet was dissolved in 30 μl buffer mix (0.1M Tris HCl buffer with 1% BSA-20 μl, 10% trehalose-10 μl, 20% Tween-20 μl). This can be stored at 4-10° C. for 2-3 days. This solution was imbibed on the conjugate release matrix and kept for drying for 2-3 hours under controlled humidity (<20%) at 37-40° C.

0.5 mg/ml of antibodies were dispended on the test zone (polyclonal Rabbit anti-Human VWF antibody, Dako, Denmark) and control zone (secondary antibody—Anti-Rabbit IgG, Sigma, USA). The membranes were kept for drying for 2 hours under controlled humidity.

### Example 2

### Assembling and Setting up the Strip

The membranes were assembled in the order: Sample pad, Conjugate pad, Reaction matrix and absorbant pad with the overlap so as to obtain a strip. The assembled strip was then packed in a plastic cassette (4 mm diameter used).

70 μl of the sample was added on the sample pad through the cassette window. The sample ran by capillary action through the membrane to give the TEST and CONTROL line/band. The results were observed as visual detection of intense red colour band/bands within 15 minutes after sample application.

### Example 3

### Sample Application and Testing

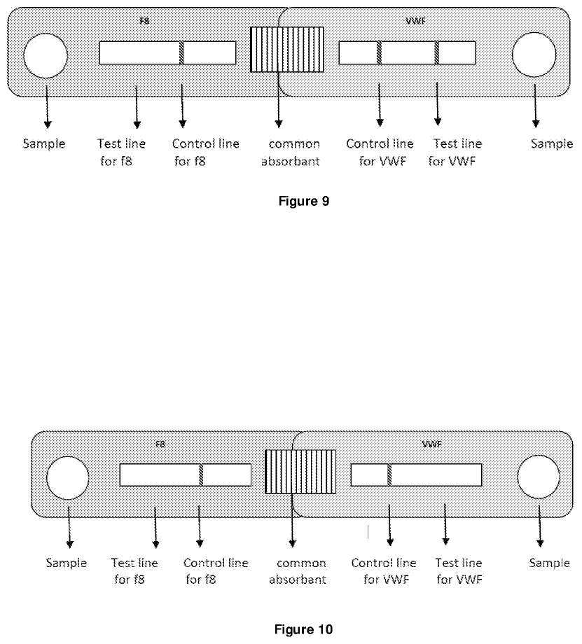

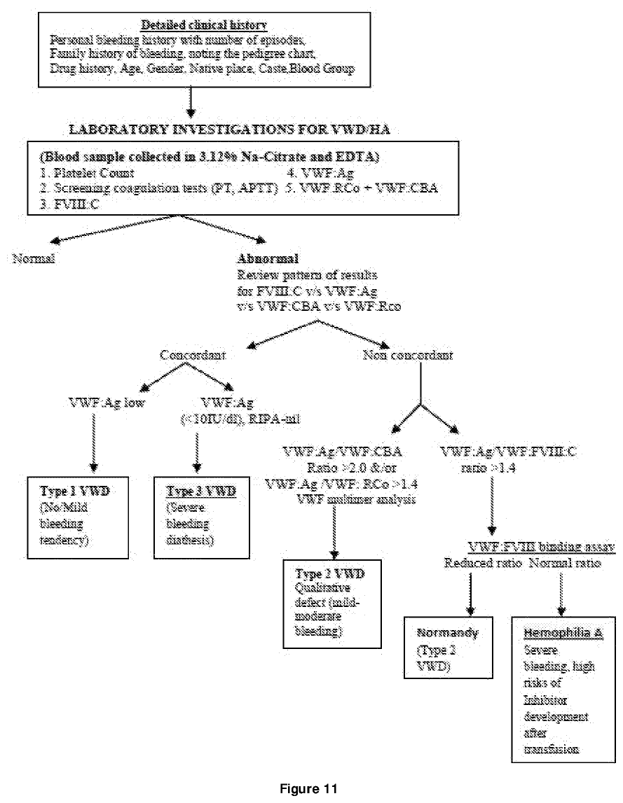

The plasma sample to be tested (fresh/stored at 4° C. for 2-3 hours/−20° C./−70° C. for months) were added to the sample window/cassette.
2. Normal sample: All four intense bands were visually observed in the strip (FIG. 8).
3. Hemophilia A sample: No test line (in case of severe/moderate Hemophilia A i.e., F8<5 IU/dL) but bright control line seen in F8 (Factor VIII) section. Both Test and control line seen in VWF section (FIG. 9).
   For mild Hemophilia A- faint to intense band (5-50 IU/dL) can be observed on the test zone depending on the F8 content in the sample.
4. Von Willebrand disease sample: No test line seen in VWF section but bright control line seen. Also, no test seen in F8 section of the kit and good control line seen (Since in Type 3 VWD-VWF and F8 both are reduced) (FIG. 10).

In an embodiment, a commercially available strip reader can be used that would directly measure the concentrations of the antigen factors from the strip. The kit is provided with photo-strip meter (i.e., dilutions of the known standard provided with the kit in the form of photo print) to compare the intensity of the patients strip to the standard provided with the kit. By comparing the intensity of the test band with the given strip meter, it becomes easy to interpret the severity of the disease when corelated with the bleeding symptoms.

Results and Comparative Data

100 VWD patients (Type 1, Type 2 and Type 3VWD) were studied on the modified standardized strip with 99% specificity and 99% accuracy (±3%) on the mdi (Advanced microdevices, Ambala, India) LFIA membranes.

TABLE 1
Comparative features of the present technique with other available techniques for the detection of VWF

| | Inhouse/ | Detection | | Cost/Test | | | |
|---|---|---|---|---|---|---|---|
| Principle | Commercial | time | | (Rs.) | Expertise | Portability | Instrument |
| LFIA (Recent | Inhouse | 15 | minutes | <40 | X | ✓ | X |
| present study) | | | | | | | |
| ELISA | Commercial | 6-8 | hours | 1000 | ✓ | X | ✓ |
| Electrophoresis | Inhouse | 24 | hours | 500* | ✓ | X | ✓ |
| Clot based | Commercial | 4-6 | hours | 4000 | ✓ | X | ✓ |
| Latex agglutination | Commercial | 1-2 | hours | 2000-3000 | ✓ | X | ✓ |

✓—yes,
x—no,
*usually done in batch processing

TABLE 2
Comparative features of the present technique with other available techniques for the detection of Factor VIII:Ag

| | Inhouse/ | Detection | | Cost/Test | | | |
|---|---|---|---|---|---|---|---|
| Principle | Commercial | time | | (Rs.) | Expertise | Portability | Instrument |
| ELISA | Commercial | 6-8 | hours | 1000 | ✓ | X | ✓ |
| Clot based | Commercial | 4-6 | hours | 2000 | ✓ | X | ✓ |
| Chromogenic- | | | | | | | |
| a. ELISA based | Commercial | 6-8 | hours | 2000 | ✓ | X | ✓ |
| b. Automated | | 2-3 | hours | 4000-5000 | | | |
| LFIA (present | In house | 15 | minutes | <40 | X | ✓ | X |
| kit) | | | | | | | |

✓—yes,
x—no

Advantages
The method of the present invention requires quite little amount of sample i.e. only 50 μL plasma and even the pediatric cases can be diagnosed easily.
There is no need of any specialized equipments like coagulometer, centrifuge, ELISA readers which are required for conventional diagnostic tests of these disorders.
The time for diagnosis is just 10 minutes after the application of plasma whereas the conventional tests take a minimum of 3-6 hours depending on the techniques used.
There is no need of any technical expertise or training to diagnose the patients with the present kit as the diagnosis is based only on visualization of bands i.e. presence or absence of bands on the strips.
The working cost of the strip is less than Rs 40 as against Rs 1000-6000 for the conventional diagnostic tests.
Simultaneous detection of both VWF as well as FVIII protein can be carried out at the same time using the multiplex kit.

1. A point-of-care apparatus, wherein the apparatus is in the form of a lateral flow immunoassay-based strip for detection of FVIII and/or Von Willebrand factor (VWF) in a sample comprising:
a) a sample pad;
b) a conjugate pad;
c) a reaction matrix and
d) a common absorbant pad
wherein the strip is fixed in a plastic cassette and comprises gold nanoparticles in the size range of 10-20 nm, primary antibodies and/or secondary antibodies specific to VWF/FVIII and lateral flow grade membranes.

2. The apparatus as claimed in claim 1, wherein the apparatus is used for separately or simultaneously detecting VWF and/or FVIII.

3. The apparatus as claimed in claim 1, wherein the sample pad is made up of glass fiber or polyester membrane with varying thickness of 0.35 mm-0 6 mm and is optionally pretreated with a buffer and a detergent.

4. The apparatus as claimed in claims 1-3, wherein the buffer is selected from Phosphate buffered saline (PBS), Borate or Tris buffer with varying content of blocking agents for enhancing the stability and releasing the gold conjugate from the conjugate pad, ensuring the uniform movement of gold nanoparticles tagged antibodies and samples on the strip.

5. The apparatus as claimed in claim 4, wherein the blocking agent is selected from bovine serum albumin (BSA), Polyethylene glycol (PEG) and gelatin.

6. The apparatus as claimed in claim 1, wherein the apparatus is kept in a sealed aluminium pouch with the desiccant between 4-10° C.

7. The apparatus as claimed in claim 1, wherein the conjugate pad comprises a membrane made up of a material selected from glass fiber, cellulose filters and surface treated polyester or polypropylene filters capable of releasing the conjugate after drying on said membrane.

8. The apparatus as claimed in claim 7, wherein the membranes are optionally treated with one or more blocking agents, buffers and detergents to maintain a pH between 7-9 with a wicking time between 18-48 seconds.

9. The apparatus as claimed in claim 1, wherein the conjugate comprises antibodies conjugated with gold nanoparticles.

10. The apparatus as claimed in claim 1, wherein the reaction matrix comprises nitrocellulose (NC), cellulose or nitrocellulose acetate membranes of pore sizes between 5 μ to 15 μ.

11. A uniplex or multiplex kit for detection of Von Willebrand factor (VWF) and/or FVIII in a sample comprising:
(i) A strip as claimed in claims 1-10;
(ii) A photo-strip meter or reader that would directly measure the VWF and/or FVIII concentrations from the strip; and
(iii) an instruction/operation manual.

12. The kit as claimed in claim 11, wherein the strip reader has dilutions of the known standard in the form of photo print to compare the intensity of the patients strip to the standard provided with the kit.

13. A method for preparing the point-of-care apparatus as claimed in claims 1-10, comprising:
a) preparing a sample pad by pretreating a glass fiber or polyester membrane with a buffer and a detergent;
b) conjugating gold nanoparticles (GNPs) with antibodies to make the conjugate pad;
c) selecting a reaction matrix of pore size between 5 μ to 15 μ and an absorbant pad made of high-quality cellulose fiber; and
d) assembling the membranes in the order: Sample pad, Conjugate pad, Reaction matrix and absorbant pad with the overlap so as to obtain a strip and packing the same in a plastic cassette, in an aluminium pouch with a dessicant.

14. A lateral flow immunoassay based (LFIA) method for detection of Hemophilia and/or Von Willebrand Factor (VWF) in a sample comprising:
a) adding about 50-700 μ of the sample on the sample pad (1) through a cassette window (2) with the help of dropper provided with the kit or pipette;
b) allowing the sample to run by capillary action through the membrane for about 5 to 15 minutes; and
c) visually detecting a red color band.

15. The method as claimed in claim 14, wherein the sample is a plasma sample from a subject.
